(19) United States
(12) Patent Application Publication
(10) Pub. No.: US 2026/0007305 A1
(43) Pub. Date: Jan. 8, 2026

(54) STEERABLE ENDOSCOPE DEVICE

(71) Applicant: UNOSCOPE TECHNOLOGY INC., Taichung City (TW)

(72) Inventors: Li-Te Yang, Taichung City (TW); Chien-Hsien Yeh, Taichung City (TW); Yen-Cheng Chen, Taichung City (TW)

(73) Assignee: UNOSCOPE TECHNOLOGY INC., Taichung City (TW)

(21) Appl. No.: 19/001,382

(22) Filed: Dec. 24, 2024

(30) Foreign Application Priority Data
Jul. 5, 2024 (TW) 113125406

(51) Int. Cl. A61B 1/005; A61B 1/00; A61B 1/018; A61B 1/04; A61B 1/303

(52) U.S. Cl. CPC A61B 1/0051; A61B 1/00105; A61B 1/018; A61B 1/042; A61B 1/303

(57) ABSTRACT

A steerable endoscope device with capability of reducing discomfort of a participant during an examination includes a first tube member and a second tube member. The first tube member has a first tunnel. The second tube member is disposed in the first tunnel in a stretchable and retractable manner relative to the first tube member and includes an extending portion and a deformation portion. The extending portion is extended along an axis, and the deformation portion is connected to the extending portion. When the second tube member is retracted to a position where the deformation portion is located in the first tunnel, a wall of the first tunnel constrains an orientation of the deformation portion to be parallel to the axis. When the second tube member is extended to a position where the deformation portion is exposed from the first tunnel, the deformation portion deforms and deviates from the axis.

### BACKGROUND OF THE DISCLOSURE

### 1. Field of the Disclosure

The present invention relates generally to an endoscope device, and more particularly to one that is steerable to achieve variation of the orientation of the endoscope device.

### 2. Description of the Prior Art

Recently, the incidence of endometrial cancer has been increasing year by year, and a hysteroscopy is primarily used for evaluating uterine surgeries and diagnosing issues related to heavy bleeding, severe cramping, frequent miscarriages, or concerns associated with suspected endometrial cancer. However, when using the hysteroscopy to examine the uterus, the hysteroscope passes sequentially through the vagina, cervical opening, and cervix into the uterine cavity. The narrowest part is the cervical opening to the cervix, which poses a higher risk of perforation or discomfort, especially in nulliparous patients. Therefore, designing a hysteroscope that reduces the risk of perforation or discomfort during examination has become a major challenge in the industry.

Other endoscopes, such as flexible ureteroscopes and bronchoscopes, incorporate bending mechanisms to provide steering capabilities for the endoscope's tip. However, these mechanisms require numerous components to function, often resulting in complex structures, reduced strength, limited internal space, and higher costs. If a simpler mechanism could replace the complex flexible tube structure, it would present broader application opportunities.

### SUMMARY OF THE DISCLOSURE

The present invention provides a steerable endoscope that reduces the risk of perforation or discomfort during examination for solving the above drawbacks.

According to an embodiment, a steerable endoscope device includes a first tube member and a second tube member. The first tube member has a first tunnel. The second tube member is disposed in the first tunnel in a manner stretchable and retractable relative to the first tube member. The second tube member includes an extending portion and a deforming portion. The extending portion is extended along an axis. The deforming portion is connected to the extending portion. When the second tube member is retracted relative to the first tube member to a position that the deforming portion is contained in the first tube member, a wall of the first tube member constrains an orientation of the deforming portion to be substantially parallel to the axis. When the second tube member is stretched relative to the first tube member to a position that the deforming portion is exposed from the first tube member, the deforming portion deviates from the axis due to deformation.

According to an embodiment, when the second tube member is stretched relative to the first tube member to the position that the deforming portion is exposed from the first tube member, an end of the deforming portion and an end of the first tube member is included by an angle, and the angle is greater than 10 degrees.

According to an embodiment, the second tube member further includes a second tunnel communicating with the first tunnel, and the endoscope further includes a base connected to the first tube member. The base has an instrument passage for allowing an instrument to pass therethrough, such that the instrument enters a body cavity through the first tunnel and the second tunnel.

According to an embodiment, the second tube member further includes a second tunnel communicating with the first tunnel. The endoscope further includes a base connected to the first tube member. The base has a water passage for allowing an object to pass therethrough, such that the object enters a body cavity through the first tunnel and the second tunnel.

According to an embodiment, the second tube member further includes a second tunnel communicating with the first tunnel, and the endoscope further includes an image capturing module disposed on an end of the deforming portion. The image capturing module is utilized for capturing an image inside a body.

According to an embodiment, the endoscope device further includes a base connected to the first tube member. The base has a signal passage communicating with at least one of the first tunnel and the second tunnel. The signal passage allows a signal wire of the image capturing module to pass therethrough, such that the signal wire is connected to an image display device through the signal passage and at least one of the second tunnel and the first tunnel.

According to an embodiment, a hardness of the first tube member is greater than a hardness of the second tube member, and the endoscope device further includes a base. The first tube member is secure to the base, and the second tube member is disposed in the first tunnel of the first tube member in a movable manner.

According to an embodiment, the endoscope device further includes a push button disposed on the second tube member. The push button is for providing a force application point, such that the second tube member is movable relative to the first tube member.

According to an embodiment, a hardness of the first tube member is greater than a hardness of the second tube member, and the endoscope device further includes a base. The second tube member is secure to the base, and the first tube member sheathes on the second tube member in a movable manner.

According to an embodiment, the endoscope device further includes a push button disposed on the first tube member. The push button is for providing a force application point, such that the first tube member is movable relative to the second tube member.

According to an embodiment, a bore diameter of the first tunnel is substantially equal to a tube diameter of the second tube member.

According to an embodiment, a steerable endoscope device includes a first tube member and a second tube member. The first tube member has a first tunnel and a guiding portion, and the guiding portion is disposed at a position near an opening of the first tunnel of the first tube member. The second tube member is disposed in the first tunnel in a manner stretchable and retractable relative to the first tube member. When the second tube member is retracted in the first tunnel of the first tube member, an orientation of the second tube member is identical to an orientation of the first tube member. When the second tube member is stretched relative to the first tube member, the guiding portion of the first tube member guides a portion of the second tube member that is stretched out of the first tube member to deviate from the orientation of the first tube member.

According to an embodiment, a steerable endoscope device includes a first tube member and a second tube member. The first tube member has at least one first tunnel, and the first tube member includes a main body portion and a flexible portion. The main body portion is extended along a central line. The flexible portion is connected to the main body, wherein the at least one first tunnel penetrates the main body portion and the flexible portion. The second tube member is disposed in the at least one first tunnel in a movable manner. When the second tube member is moved along the at least one first tunnel of the first tube member to a position that the tube member is corresponding to the flexible member, the second tube member forces an orientation of the flexible portion to be substantially parallel to the central line of the main body portion. When the second tube member is moved along the at least one first tunnel of the first tube member to a position that the tube member departs from the flexible member, the second tube member does not constrain the flexible portion, such that the orientation of the flexible portion deviates from the central line of the main body portion.

In summary, the endoscope device of the present invention includes the stretchable and retractable tube assembly formed by the first tube member and the second tube member that can extend and retract relative to each other. In this arrangement, the hardness of the second tube member (inner tube) is less than that of the first tube member (outer tube). Practically, the material at the front end of the second tube member (inner tube) can be the flexible shape memory plastic or the flexible elastomer, allowing the portion of the second tube member to form the deformable portion. In this manner, when the second tube member retracts relative to the first tube member to the position where the deforming portion collapses in the first tunnel, the wall of the first tunnel restricts the orientation of the deforming portion to be substantially parallel to the axis. Consequently, the bore diameter of the first tunnel of the first tube member in the present invention can be substantially equal to the tube diameter of the second tube member, aiding in the control of the overall outer diameter of the endoscope device (i.e., the stretchable and retractable tube assembly). This helps reduce the risk of penetrating the affected area or causing discomfort during the examination of the uterine cavity.

Additionally, when the second tube member extends relative to the first tube member to expose the deforming portion out of the first tube member, the deforming portion can be deformed and deviate from the axis. In this manner, when the second tube member of the endoscope device of the present invention enters the uterine cavity, the deformable portion that deviates from the axis can capture images of the uterine cavity walls from a more comprehensive perspective, thereby enhancing the accuracy of the detection.

In summary, this steerable endoscope device can reduce the discomfort experienced by patients during examinations and lower the risk of penetrating the affected area with the uterine endoscope, thereby enhancing the comfort and safety of the patients.

### BRIEF DESCRIPTION OF THE DRAWINGS

The present invention will be best understood by referring to the following detailed description of one illustrative embodiment in conjunction with the accompanying drawings, in which

FIG. 1 is a diagram of an endoscope device in the working environment according to an embodiment of the present invention;

FIG. 2 is a diagram of the endoscope device according to the embodiment of the present invention;

FIG. 3 is an exploded diagram of the endoscope device according to the embodiment of the present invention;

FIG. 4 is a cross-sectional view of the endoscope device in a contained status according to the embodiment of the present invention;

FIG. 5 is a cross-sectional view of the endoscope device in the contained status according to the embodiment of the present invention;

FIG. 6 is a diagram of the endoscope device in a use status according to the embodiment of the present invention;

FIG. 7 is a cross-sectional view of an endoscope device according to another embodiment of the present invention;

FIG. 8 is a partly cross-sectional view of an endoscope device in a contained status according to another embodiment of the present invention;

FIG. 9 is a partly cross-sectional view of the endoscope device in a use status according to another embodiment of the present invention;

FIG. 10 is a partly cross-sectional view of an endoscope device in a contained status according to another embodiment of the present invention; and

FIG. 11 is a partly cross-sectional view of the endoscope device in a use status according to another embodiment of the present invention.

### DETAILED DESCRIPTION

To facilitate understanding of the present invention for those skilled in the art, multiple embodiments of the present invention will be explained below, along with the drawings which illustrate the composition of the present invention and the intended effect to be achieved in detail. It should be noted that, in order to provide a more straightforward description of the basic structure or method of the implementation of the present invention, the accompanying drawings are simplified schematic views and, therefore, only show components and combinations relevant to the present invention. The actual components, layouts, and arrangements may be more complicated. In addition, for the sake of illustration, the components shown in each of the accompanying drawings of the present invention are not drawn to exact scale with the actual number, shape, and size they are in practical implementations, and their actual scale may be adjusted as required.

The directional terms, such as “up”, “down”, “left”, “right”, “front”, and “rear”, mentioned in the following embodiments simply indicate the directions in the accompanying drawings. Therefore, the directional terms used in the descriptions of the present invention are for illustrative purposes, and are not limitations of the present invention.

Though the wordings “first”, “second”, “third”, etc., can be used to indicate multiple components, the components are not limited by these terms. Such wordings are merely used for distinguishing one component from other components in the specification. Following the sequence of being recited, the language in claims may use different orderings to describe the same components. In other words, a “first some component” mentioned in the following descriptions may be a “second some component” in the claims.

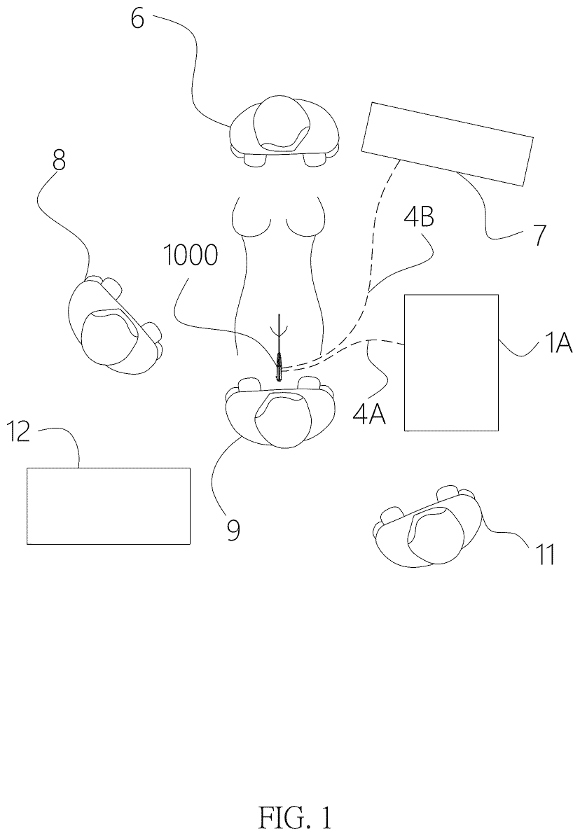

Please refer to FIG. 1. FIG. 1 is a diagram of an endoscope device 1000 in the working environment according to an embodiment of the present invention. The endoscope device 1000 is connected to a screen 7 via a signal line 4B, and through a water route 4A to a water injection device 1A. In practice, after the patient changes clothes and lies on the examination table, the doctor 9 removes the vaginal dilator from the instrument holding rack 12 and places it in the patient's vagina while disinfecting the cervix. Once the cervix is disinfected, the doctor 9 inserts the endoscope device 1000 through the vagina and cervix into a uterine cavity, and injects a substance (saline) from the water injection device 1A into the uterine cavity via the water route 4A to expand the uterine cavity. The endoscope device 1000 then transmits the captured images to the screen 7 through the signal line 4B, allowing the doctor 9 to perform the examination with the endoscope device 1000 while utilizing the image feedback provided by the endoscope device 1000 to enhance the precision and safety of the procedure. It is worth mentioning that the endoscope device 1000 of this invention is preferably applied to a hysteroscope device, but this invention is not limited to the present application; for example, the endoscope device 1000 can also be used in an endoscope device, such as a cystoscope.

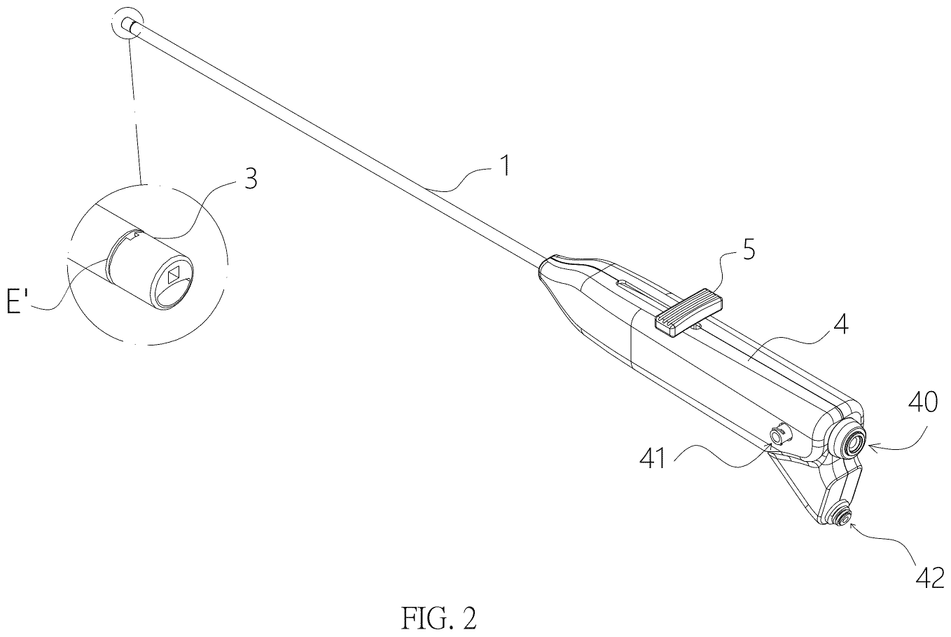

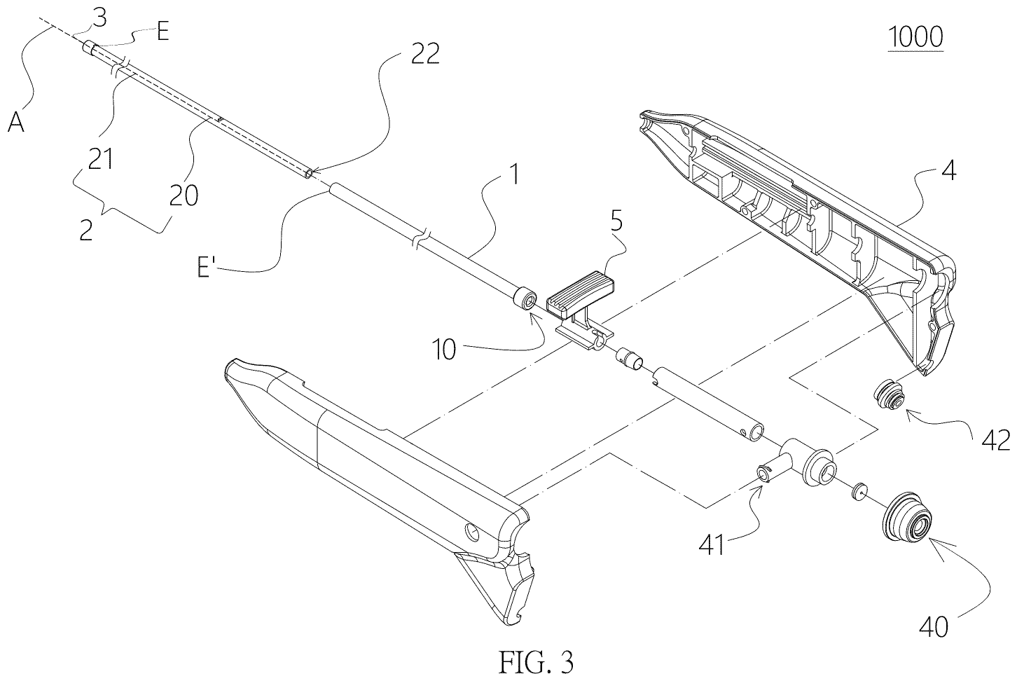

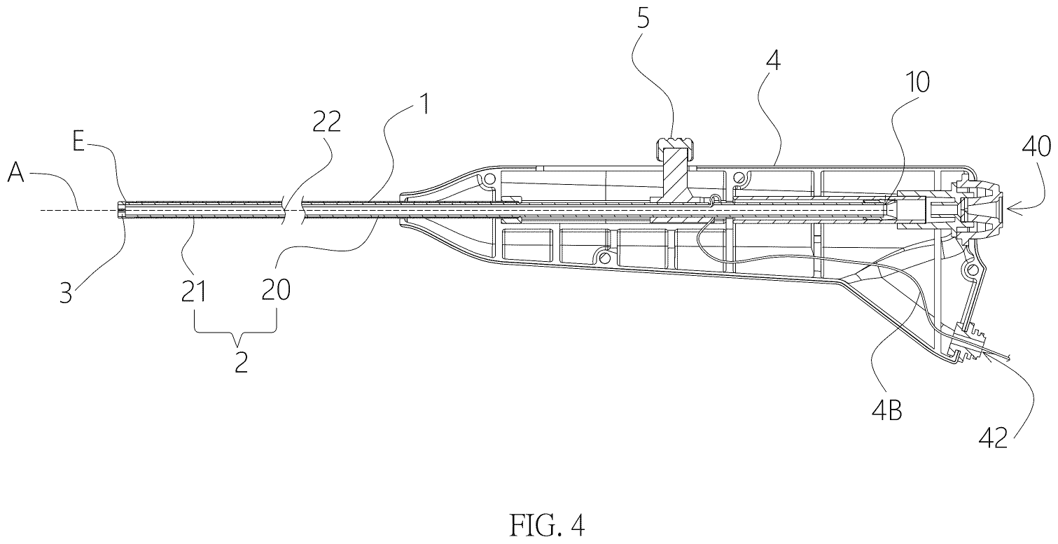

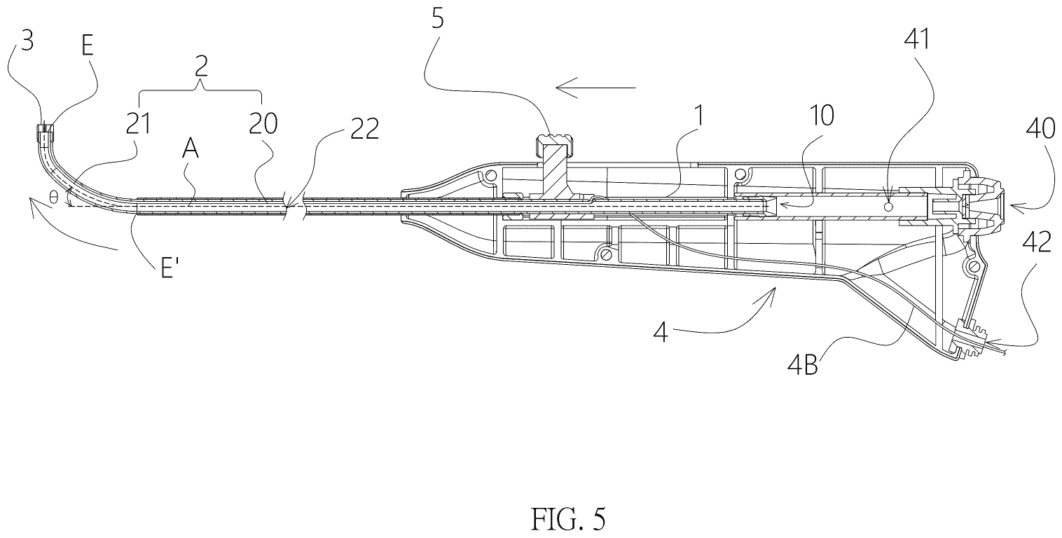

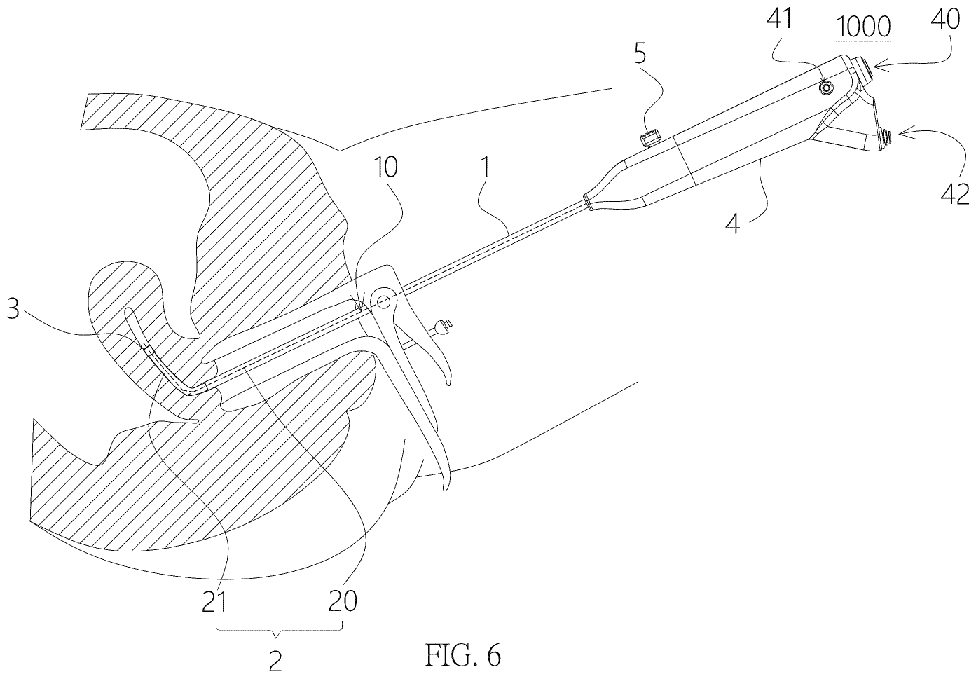

Please refer to FIG. 2 to FIG. 6. FIG. 2 is a diagram of the endoscope device 1000 according to the embodiment of the present invention. FIG. 3 is an exploded diagram of the endoscope device 1000 according to the embodiment of the present invention. FIG. 4 is a cross-sectional view of the endoscope device 1000 in a contained status according to the embodiment of the present invention. FIG. 5 is a cross-sectional view of the endoscope device 1000 in the contained status according to the embodiment of the present invention. FIG. 6 is a diagram of the endoscope device 1000 in a use status according to the embodiment of the present invention. As shown in FIG. 2 to FIG. 6, the steerable endoscope device 1000 includes a first tube member 1, a second tube member 2 and an image capturing module 3. The first tube member 1 has a first tunnel 10. The second tube member 2 is disposed in the first tunnel 10 in a manner stretchable and retractable relative to the first tube member 1, i.e., the endoscope device 1000 of the present invention is a stretchable and retractable tube assembly included by the first tube member 1 and the second tube member 2. The first tube member 1 is an outer tube of the stretchable and retractable tube assembly, and the second tube member 2 is an inner tube of the stretchable and retractable tube assembly. The second tube member 2 includes an extending portion 20 and a deforming portion 21. The extending portion 20 is extended along an axis A. The deforming portion 21 is connected to the extending portion 20. The second tube member 2 further includes a second tunnel 22 communicating with the first tunnel 10 of the first tube member 1. The image capturing module 3 is disposed on an end E of the deforming portion 21 of the second tube member. The image capturing module 3 is utilized for capturing images within the uterine cavity.

In practice, the material at a front end of the second tube member 2 (inner tube) may be a flexible shape memory plastic or a flexible elastomer, allowing a portion of the second tube member 2 to be formed into the deforming portion 21 through the shape memory characteristics of the flexible shape memory plastic or the flexible elastomer by a pre-processing method (such as local heating). In this manner, when the second tube member 2 is retracted relative to the first tube member 1 such that the deforming portion 21 retracts within the first tunnel 10, the deforming characteristics of the deforming portion 21 allow the wall of the first tunnel 10 to force the deforming portion 21 to deform, thereby restricting the direction of the deforming portion 21 to be substantially parallel to axis A during the retraction process of the second tube member 2 relative to the first tube member 1. Consequently, the diameter of the first tunnel 10 of the first tube member 1 only needs to accommodate the space of the deforming portion 21, which is restricted and oriented substantially parallel to axis A (as shown in FIG. 4). In other words, a bore diameter of the first tunnel 10 of the first tube member 1 can be substantially equal to a tube diameter of the second tube member 2 of the endoscope device 1000 in the present invention.

Furthermore, when the second tube member 2 extends relative to the first tube member 1 such that the deforming portion 21 is exposed from the first tube member 1, the deforming portion 21 deforms and deviates from axis A (as shown in FIG. 5). In this manner, when the second tube member 2 of the endoscope device 1000 in the present invention enters the uterine cavity, the deforming portion 21, which is deviated from axis A, can capture images of the walls of the uterine cavity in a more comprehensive manner, thereby enhancing the precision of the inspection.

As shown in FIG. 5, when the second tube member 2 extends relative to the first tube member 1 such that the deforming portion 21 is exposed from the first tube member 1, a line connecting the end E of the deforming portion 21 and an end E′ of the first tube member 1 forms an angle θ with the axis A. In this embodiment, the angle θ is 20 degrees, but the angle θ of the present invention is not limited thereto. The angles θ greater than 10 degrees when the deforming portion 21 of the second tube member 2 is exposed from the first tube member 1 are within the scope of the present invention.

In this embodiment, the endoscope device 1000 further includes a base 4, which is connected to the first tube member 1. Additionally, the base 4 has an instrument passage 40. The instrument passage 40 is in communication with the first tunnel 10 of the first tube member 1 and is utilizing for allowing a surgical instrument (not shown in the figures) to pass through. In practice, when the second tube member 2 is inserted into the uterine cavity, the surgical instrument can enter the uterine cavity through the instrument passage 40, the first tunnel 10, and the second tunnel 22, ultimately reaching the uterine cavity for surgical procedures such as resection and sampling.

In this embodiment, the surgical instrument can be a biopsy forceps, a rat tooth alligator grasper, a lasso, or scissors, among others, but the present invention is not limited to the instruments described in this embodiment. Any surgical instruments that may be used during the surgical procedure are within the scope of the present invention.

In this embodiment, the base 4 further includes a water passage 41, which is in communication with the first tunnel 10 of the first tube member 1. Additionally, the water passage 41 is also connected to the water route 4A as shown in FIG. 1, allowing an object (saline) from the water injection device 1A to pass through the water route 4A and into the water passage 41. Thus, when the second tube member 2 is inserted into the uterine cavity, the object (saline) from the water injection device 1A can be delivered to the uterine cavity through the water route 4A, the water passage 41 of the base 4, the first tunnel 10 of the first tube member 1, and the second tunnel 22 of the second tube member 2. In this manner, the doctor 9 can inject the object (saline) from the water injection device 1A into the uterine cavity while conducting an examination with the endoscope device 1000, allowing the cavity to be expanded for further examination by the doctor. In this embodiment, the object (saline) can be carbon dioxide (CO2) or physiological saline, among others, as determined by the doctor 9 for injection, but the present invention is not limited thereto.

In this embodiment, the base 4 further includes a signal passage 42, which communicates with at least one of the first tunnel 10 of the first tube member 1 and the second tunnel 22 of the second tube member 2 and is used for the signal line 4B of the image capturing module 3 to pass through. Thus, the signal line 4B can connect the image capturing module 3 to the screen 7, as shown in FIG. 1, via at least one of the second tunnel 22 and the first tunnel 10, and the signal passage 42. In this manner, the doctor 9 can conduct the examination with the endoscope device 1000 while utilizing the screen 7 to display the image feedback provided by the image capturing module 3, allowing for the simultaneous operation of the surgical instruments, thereby improving the precision and safety of the procedure.

In this embodiment, a hardness of the first tube member 1 of the endoscope device 1000 is greater than the hardness of the second tube member 2, and the second tube member 2 (the inner tube) is configured to be movable within the first tunnel 10 of the first tube member 1 (the outer tube). The first tube member 1 (the outer tube) is fixed to the base 4, while the second tube member 2 (the inner tube) is designed for mobility. The greater hardness of the first tube member 1 provides higher rigidity, offering a stable support structure that enhances stability and control of the overall device during operation. In contrast, the smaller hardness of the second tube member 2 allows for better flexibility, especially at its deforming portion 21, enabling it to more easily adapt to the curves and changes from the cervical canal to the uterine cavity due to the properties of the flexible material. When the endoscope device 1000 is used for examination, the second tube member 2 can freely extend and retract relative to the first tube member 1, utilizing its lower hardness to reduce irritation to the walls of the uterine cavity during entry, thereby minimizing discomfort for the subject. At the same time, the greater hardness of the first tube member 1 ensures the structural stability of the overall device, preventing bending or deformation during operation, thus enhancing accuracy and safety.

In this embodiment, the endoscope device 1000 further includes a push button 5 disposed on the second tube member 2. The push button 5 serves as a force application point, allowing the user or doctor 9 to move the second tube member 2 relative to the first tube member 1 by operating the push button 5. In this manner, the user or doctor 9 can apply an appropriate amount of force through the push button 5 to adjust the position of the second tube member 2 in relation to the first tube member 1.

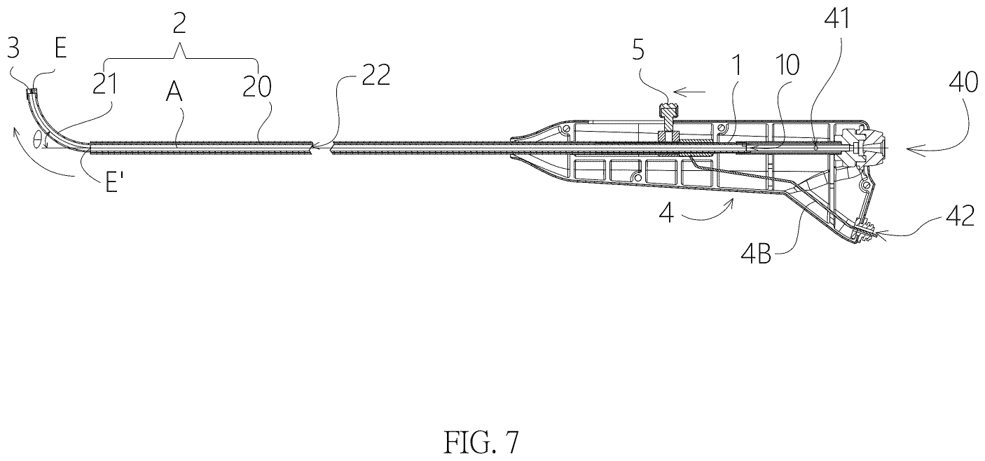

It is worth mentioning that the stretchable and retractable tube assembly of the endoscope device 1000 in the present invention is not limited to the second tube member 2 (the inner tube) being configured to be movable within the first tube member 1 (the outer tube). Please refer to FIG. 7. FIG. 7 is a cross-sectional view of an endoscope device 1000′ according to another embodiment of the present invention. The primary difference between the endoscope device 1000′ and the aforementioned endoscope device 1000 is that the first tube member 1 (the outer tube) of the endoscope device 1000′ is configured to be movable over the second tube member 2 (the inner tube), meaning that the first tube member 1 (the outer tube) in endoscope device 1000′ has a movable design, while the second tube member 2 (the inner tube) is fixed to the base 4. Depending on practical demands, the present invention can adopt the stretchable and retractable tube assembly of the endoscope device 1000, where the first tube member 1 (the outer tube) is fixed to the base 4, and the second tube member 2 (the inner tube) is designed to be movable, or it can apply the stretchable and retractable tube assembly of endoscope device 1000′, where the first tube member 1 (the outer tube) is designed to be movable, and the second tube member 2 (the inner tube) is fixed to the base 4.

In this embodiment, because the deforming portion 20 of the second tube member 2 is designed to be flexible, a bore diameter of the first tunnel 10 of the first tube member 1 can be substantially identical to a tube diameter of the second tube member 2. When the second tube member 2 is retracted into the first tunnel 10, the deforming portion 21 will be tightly constrained by the wall of the first tunnel 10 and will be oriented substantially parallel to the axis A. This design allows the bore diameter of the first tunnel 10 of the outer tube (i.e., the first tube member 1) of the endoscope device 1000 to only need to provide a dimension that is essentially the same as the tube diameter of the inner tube (i.e., the second tube member 2), without needing to accommodate space for the deforming portion 20 of the second tube member 2 to deform (i.e., the deforming portion 20 of the second tube member 2 only deforms when it extends out of the first tunnel 10). Therefore, the flexible design of the deforming portion 20 of the second tube member 2 of the present invention can effectively limit the overall size of the endoscope device 1000, helping to reduce irritation and discomfort for the subject when inserting into the uterine cavity. Additionally, the flexible design of the deforming portion 20 of the second tube member 2 allows the deforming portion 20 to deviate from the axis A when the second tube member 2 extends out of the first tunnel 10. In this manner, the flexible design of the deforming portion 20 of the second tube member 2 not only optimizes the overall size of the endoscope device 1000 but also provides sufficient flexibility and functionality during operation.

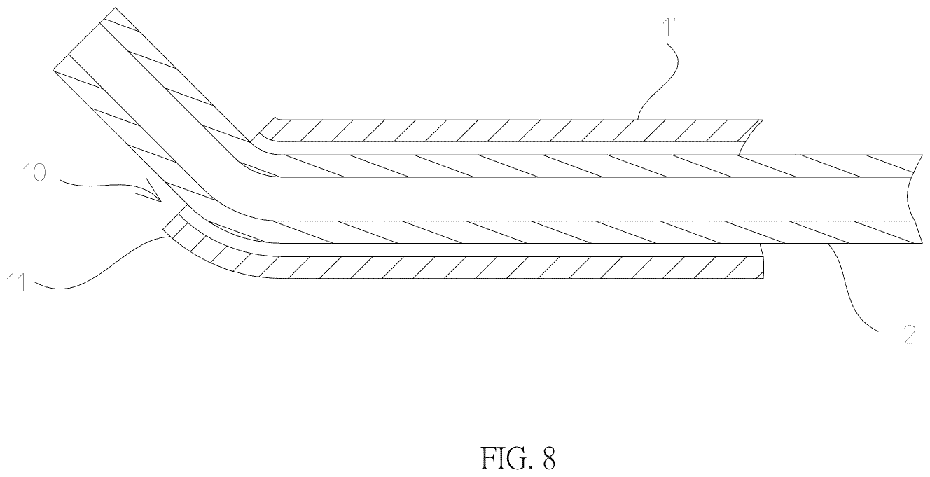

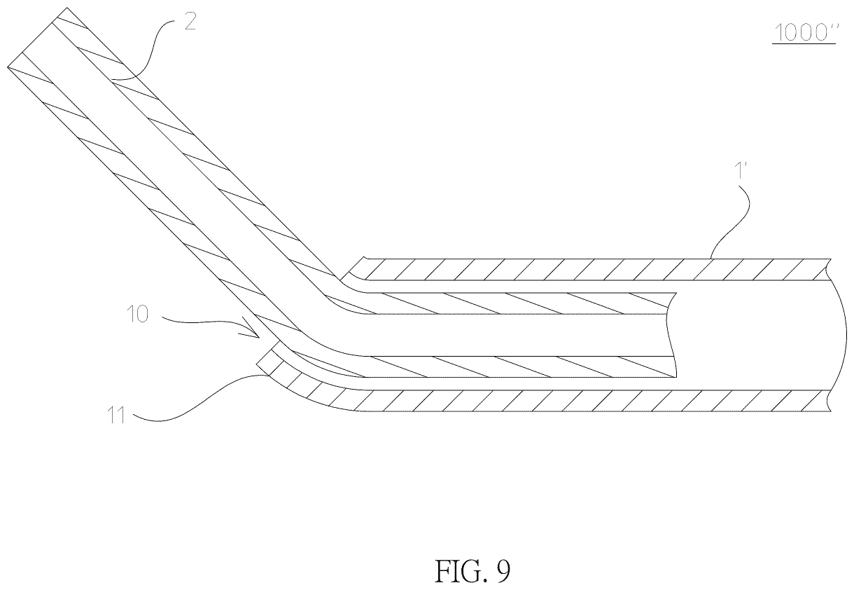

Please refer to FIG. 8 and FIG. 9. FIG. 8 is a partly cross-sectional view of an endoscope device 1000″ in a contained status according to another embodiment of the present invention. FIG. 9 is a partly cross-sectional view of the endoscope device 1000″ in a use status according to another embodiment of the present invention. The main difference between the endoscope device 1000″ and the aforementioned endoscope device 1000 is that a first tube member 1′ of the endoscope device 1000″ has a guiding portion 11, and the guiding portion 11 is disposed on the wall of the first tube member 1′ near the opening of the first tunnel 10. In such a manner, as shown in FIG. 8, when the second tube member 2 retracts into the first tube member 1′ within the first tunnel 10, the orientation of the second tube member 2 is substantially identical to that of the first tube member 1′; whereas when the second tube member 2 extends relative to the first tube member 1′, the guiding portion 11 of the first tube member 1′ can guide the second tube member 2 to extend in a direction that deviates from the orientation of the first tube member 1′, creating an angle between the extending part of the second tube member 2 and the front end of the first tube member 1′. This configuration allows the endoscope device 1000″ to provide sufficient flexibility and functionality during operation.

In this embodiment, the endoscope device 1000″ can be a flexible elastomer. Therefore, the portion of the second tube member 2 extending from the first tube member 1′ can be guided and deformed by the properties of the flexible elastomer through the guiding portion 11 of the first tube member 1′. That is, the portion of the second tube member 2 extending from the first tube member 1′ is deformed by the guiding portion 11 of the first tube member 1′, without the need for a predetermined manufacturing process (such as localized heating) to create a deformable portion in which the second tube member 2 extends from the first tube member 1′.

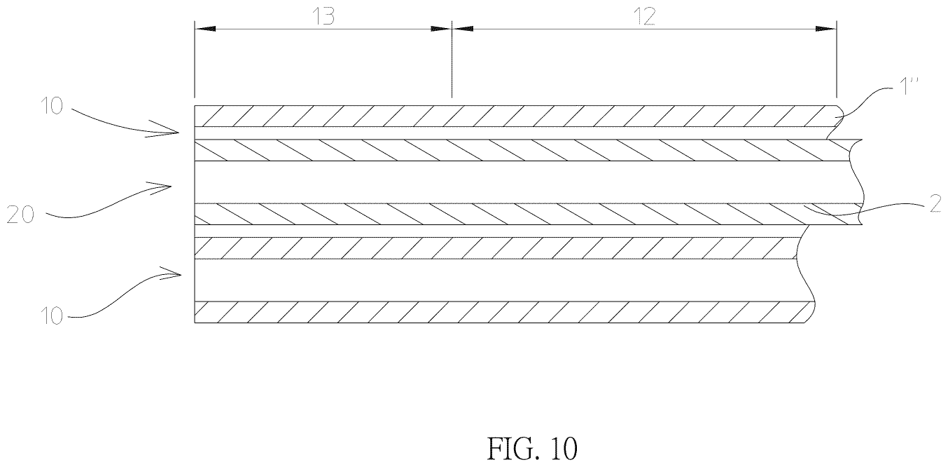

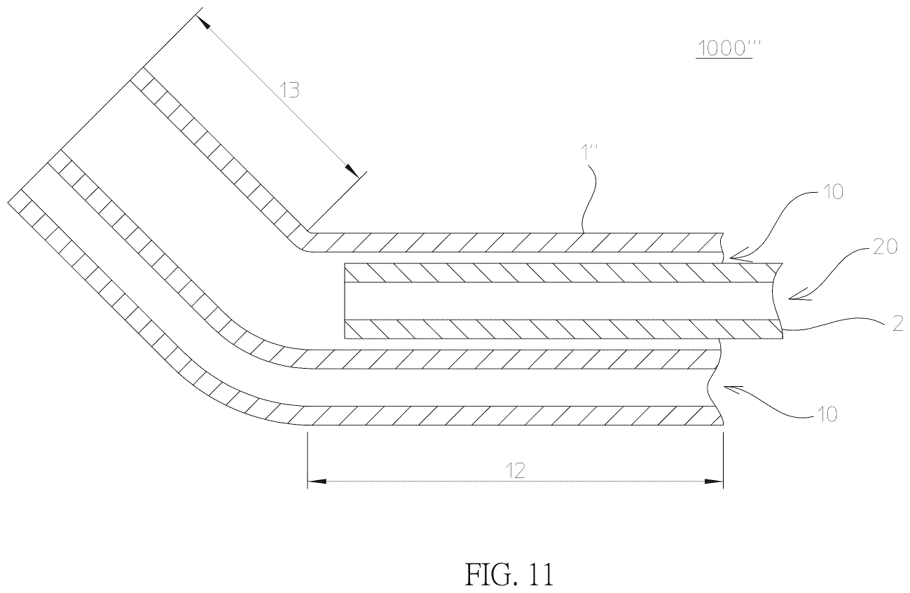

Please refer to FIG. 10 and FIG. 11. FIG. 10 is a partly cross-sectional view of an endoscope device 1000′″ in a contained status according to another embodiment of the present invention. FIG. 11 is a partly cross-sectional view of the endoscope device 1000′″ in a use status according to another embodiment of the present invention. The main difference between the endoscope device 1000″ and the aforementioned endoscope device 1000 is that the first tube member 1″ of the endoscope device 1000′″ has at least one first tunnel 10. In addition, the first tube member 1″ includes a main body portion 12 and a flexible portion 13, where the main body portion 12 extends along a central line CL, and the flexible portion 13 connects to the main body portion 12, such that the at least one first tunnel 10 of the first tube member 1″ passes through both the main body portion 12 and the flexible portion 13. Furthermore, the second tube member 2 is disposed in the at least one first tunnel 10 in a movable manner. In this embodiment, the first tube member 1″ has two first tunnels 10, which can be used respectively as an instrument passage, a water passage, or a passage for the second tube member 2. The number of the first tunnels 10 in the first tube member 1″ is not limited to the configuration depicted in this embodiment. For example, the number of first tunnels 10 in the first tube member 1″ could also be three or four, depending on practical requirements.

In this embodiment, the hardness of the material of the second tube member 2 may be greater than that of the material of the first tube member 1″. Additionally, the second tube member 2 can be a hollow tube or a solid rod, depending on practical requirements. In practice, the material at the front end of the first tube member 1″ (outer tube) can be a flexible shape memory plastic or a flexible elastomer, allowing a portion of the first tube member 1″ to be formed into a bendable flexible portion 13 through the shape memory characteristics of the flexible shape memory plastic or the flexible elastomer via a predetermined manufacturing process (such as localized heating).

In such a manner, when the second tube member 2 moves in the first tunnel 10 relative to the first tube member 1″ to a position corresponding to the flexible portion 13, the second tube member 2 can force the orientation of the flexible portion 13 of the first tube member 1″ to be substantially parallel to the central line CL of the main body portion 12. Conversely, when the second tube member 2 moves away from the position corresponding to the flexible portion 13 in the first tunnel 10, it does not constrain the flexible portion 13 of the first tube member 1″. In this way, the flexible portion 13 can deviate from the central line CL of the main body portion 12 due to the shape memory characteristics of the flexible shape memory plastic or the bending characteristics formed during the predetermined manufacturing process.

Compared to the prior art, the endoscope device of the present invention includes the stretchable and retractable tube assembly formed by the first tube member and the second tube member that can extend and retract relative to each other. In this arrangement, the hardness of the second tube member (inner tube) is less than that of the first tube member (outer tube). Practically, the material at the front end of the second tube member (inner tube) can be the flexible shape memory plastic or the flexible elastomer, allowing the portion of the second tube member to form the deformable portion. In this manner, when the second tube member retracts relative to the first tube member to the position where the deforming portion collapses in the first tunnel, the wall of the first tunnel restricts the orientation of the deforming portion to be substantially parallel to the axis. Consequently, the bore diameter of the first tunnel of the first tube member in the present invention can be substantially equal to the tube diameter of the second tube member, aiding in the control of the overall outer diameter of the endoscope device (i.e., the stretchable and retractable tube assembly). This helps reduce the risk of penetrating the affected area or causing discomfort during the examination of the uterine cavity.

Additionally, when the second tube member extends relative to the first tube member to expose the deforming portion out of the first tube member, the deforming portion can be deformed and deviate from the axis. In this manner, when the second tube member of the endoscope device of the present invention enters the uterine cavity, the deformable portion that deviates from the axis can capture images of the uterine cavity walls from a more comprehensive perspective, thereby enhancing the accuracy of the detection.

In summary, this steerable endoscope device can reduce the discomfort experienced by patients during examinations and lower the risk of penetrating the affected area with the uterine endoscope, thereby enhancing the comfort and safety of the patients. The above description is merely a preferred embodiment of the present invention. Any equivalent structural variations that apply to the specification and claims of the present invention should be included within the scope of the patent for the present invention.

What is claimed is:

1. A steerable endoscope device, comprising:
a first tube member having a first tunnel; and
a second tube member disposed in the first tunnel in a manner stretchable and retractable relative to the first tube member, the second tube member comprising:
an extending portion extended along an axis; and
a deforming portion connected to the extending portion;
wherein when the second tube member is retracted relative to the first tube member to a position that the deforming portion is contained in the first tube member, a wall of the first tube member constrains an orientation of the deforming portion to be substantially parallel to the axis;
wherein when the second tube member is stretched relative to the first tube member to a position that the deforming portion is exposed from the first tube member, the deforming portion deviates from the axis due to deformation.

2. The endoscope device of claim 1, wherein when the second tube member is stretched relative to the first tube member to the position that the deforming portion is exposed from the first tube member, an end of the deforming portion and an end of the first tube member is included by an angle, and the angle is greater than 10 degrees.

3. The endoscope device of claim 1, wherein the second tube member further comprises a second tunnel communicating with the first tunnel, the endoscope further comprises:
a base connected to the first tube member, the base having an instrument passage for allowing an instrument to pass therethrough, such that the instrument enters a body cavity through the first tunnel and the second tunnel.

4. The endoscope device of claim 1, wherein the second tube member further comprises a second tunnel communicating with the first tunnel, the endoscope further comprises:
a base connected to the first tube member, the base having a water passage for allowing an object to pass therethrough, such that the object enters a body cavity through the first tunnel and the second tunnel.

5. The endoscope device of claim 1, wherein the second tube member further comprises a second tunnel communicating with the first tunnel, the endoscope further comprises:
an image capturing module disposed on an end of the deforming portion, the image capturing module being utilized for capturing an image inside body.

6. The endoscope device of claim 5, further comprising:
a base connected to the first tube member, the base having a signal passage communicating with at least one of the first tunnel and the second tunnel, the signal passage allowing a signal wire of the image capturing module to pass therethrough, such that the signal wire is connected to an image display device through the signal passage and at least one of the second tunnel and the first tunnel.

7. The endoscope device of claim 1, wherein a hardness of the first tube member is greater than a hardness of the second tube member, and the endoscope device further comprises:
a base, the first tube member being secure to the base, the second tube member being disposed in the first tunnel of the first tube member in a movable manner.

8. The endoscope device of claim 7, further comprising:
a push button disposed on the second tube member, the push button being for providing a force application point, such that the second tube member is movable relative to the first tube member.

9. The endoscope device of claim 1, wherein a hardness of the first tube member is greater than a hardness of the second tube member, and the endoscope device further comprises:
a base, the second tube member being secure to the base, the first tube member sheathing on the second tube member in a movable manner.

10. The endoscope device of claim 9, further comprising:
a push button disposed on the first tube member, the push button being for providing a force application point, such that the first tube member is movable relative to the second tube member.

11. The endoscope device of claim 1, wherein a bore diameter of the first tunnel is substantially equal to a tube diameter of the second tube member.

12. A steerable endoscope device, comprising:
a first tube member having a first tunnel and a guiding portion, the guiding portion being disposed at a position near an opening of the first tunnel of the first tube member; and
a second tube member disposed in the first tunnel in a manner stretchable and retractable relative to the first tube member;
wherein when the second tube member is retracted in the first tunnel of the first tube member, an orientation of the second tube member is identical to an orientation of the first tube member;
wherein when the second tube member is stretched relative to the first tube member, the guiding portion of the first tube member guides a portion of the second tube member that is stretched out of the first tube member to deviate from the orientation of the first tube member.

13. A steerable endoscope device, comprising:
a first tube member having at least one first tunnel, the first tube member comprising:
a main body portion extended along a central line; and
a flexible portion connected to the main body, wherein the at least one first tunnel penetrating the main body portion and the flexible portion; and
a second tube member disposed in the at least one first tunnel in a movable manner;
wherein when the second tube member is moved along the at least one first tunnel of the first tube member to a position that the tube member is corresponding to the flexible member, the second tube member forces an orientation of the flexible portion to be substantially parallel to the central line of the main body portion;
wherein when the second tube member is moved along the at least one first tunnel of the first tube member to a position that the tube member departs from the flexible member, the second tube member does not constrain the flexible portion, such that the orientation of the flexible portion deviates from the central line of the main body portion.
